(19) United States
(12) Patent Application Publication
(10) Pub. No.: US 2022/0163553 A1
(43) Pub. Date: May 26, 2022

(54) CELL PICKING DEVICE AND CELL PICKING METHOD

(71) Applicant: SHIMADZU CORPORATION, Kyoto (JP)

(72) Inventors: Mika OKADA, Kyoto (JP); Yuki KUZUHARA, Kyoto (JP); Yoshitake YAMAMOTO, Kyoto (JP); Toru EZURE, Kyoto (JP)

(73) Assignee: SHIMADZU CORPORATION, Kyoto (JP)

(21) Appl. No.: 17/531,065

(22) Filed: Nov. 19, 2021

(30) Foreign Application Priority Data
Nov. 26, 2020 (JP) 2020-196185

(51) Int. Cl. G01N 35/10; B01L 3/02; B01L 9/00; C12N 1/02; C12M 1/26; C12M 1/00; B01L 3/00

(52) U.S. Cl. CPC G01N 35/1011; B01L 3/021; B01L 9/54; C12N 1/02; B01L 2300/027; C12M 47/02; B01L 3/50855; G01N 2035/103; C12M 33/04

(57) ABSTRACT

A cell picking device that sucks cells from a liquid sample in a sample container and accommodates the sucked cells in any of a plurality of wells of an accommodating plate, includes a suction discharger that sucks cells in the sample container and discharges the sucked cells into any of the plurality of wells, the receiver that receives selection of a well in which cells are to be accommodated out of the plurality of wells of the accommodating plate as a selection well, a driver that adjusts a relative positional relationship between the suction discharger and the plurality of wells of the accommodating plate, and a work controller that controls the driver such that the suction discharger discharges cells into the received selection well out of the plurality of wells of the accommodating plate.

### BACKGROUND

### Technical Field

The present invention relates to a cell picking device and a cell picking method.

### Description of Related Art

In a case where specific cells are to be sucked from a container such as a cell culture container, a worker sucks the cells manually using a suction tool such as a pipette while checking the position of the cells with a microscope. However, since such work requires skill, an unskilled worker cannot easily suck cells. As such, a cell-sucking system that assists cell-sucking work has been suggested (see JP 2016-112012 A, for example).

In the cell-sucking system described in JP 2016-112012 A, a tubular pipette tip for sucking cells stored in a container is attached to a sucker. The position of the sucker is adjusted three-dimensionally by a transporter such that the end of the pipette tip is in close proximity to specific cells.

### SUMMARY

However, in the above-mentioned cell-sucking system, in a case where cells that have been sucked into the pipette tip are discharged into a well of a well plate, for example, a user is required to adjust the position of the well of the well plate to the position at which the pipette tip discharges cells.

An object of the present invention is to provide a cell picking device and a cell picking method that can alleviate a work load on a user when cells are accommodated in a desired well of an accommodating plate.

A cell picking device according to one aspect of the present invention sucks cells from a liquid sample in a sample container and accommodates the sucked cells in any of a plurality of wells of an accommodating plate, and may include a suction discharger that sucks cells in the sample container and discharges the sucked cells into any of the plurality of wells, the receiver that receives selection of a well in which cells are to be accommodated out of the plurality of wells of the accommodating plate as a selection well, a driver that adjusts a relative positional relationship between the suction discharger and the plurality of wells of the accommodating plate, and a work controller that controls the driver such that the suction discharger discharges cells into the received selection well out of the plurality of wells of the accommodating plate.

A cell picking method according to another aspect of the present invention of sucking cells from a liquid sample in a sample container and accommodating the sucked cells in any of a plurality of wells of an accommodating plate, may include receiving selection of a well in which cells are to be accommodated out of the plurality of wells of the accommodating plate as a selection well, sucking cells in the sample container using a suction discharger, adjusting a relative positional relationship between the suction discharger and the plurality of wells of the accommodating plate using a driver such that the cells are dischargeable into the selection well, and discharging cells that have been sucked by the suction discharger into the selection well after adjustment of the relationship by the driver.

Other features, elements, characteristics, and advantages of the present disclosure will become more apparent from the following description of preferred embodiments of the present disclosure with reference to the attached drawings.

### BRIEF DESCRIPTION OF THE DRAWING

FIG. 1 is a schematic front view showing the configuration of a cell picking device according to one embodiment of the present invention;

FIG. 2 is a schematic diagram showing an example of a selection screen displayed in a display unit of FIG. 1 when a method is created;

FIG. 3 is a schematic diagram showing an example of the selection screen displayed in the display unit of FIG. 1 when a method is created;

FIG. 4 is a schematic diagram showing an example of a selection screen displayed in the display unit of FIG. 1 when a method is created;

FIG. 5 is a block diagram showing the functional configuration of a main controller;

FIG. 6 is a flowchart showing the work of the cell picking device of FIG. 3;

FIG. 7 is a schematic diagram showing one example of a seeding state screen during picking work;

FIG. 8 is a schematic diagram showing one example of the seeding state screen during picking work; and

FIG. 9 is a schematic diagram showing one example of the seeding state screen during picking work.

### DETAILED DESCRIPTION

A cell picking device according to embodiments of the present invention will be described below in detail with reference to the drawings.

### (1) Configuration of Cell Picking Device

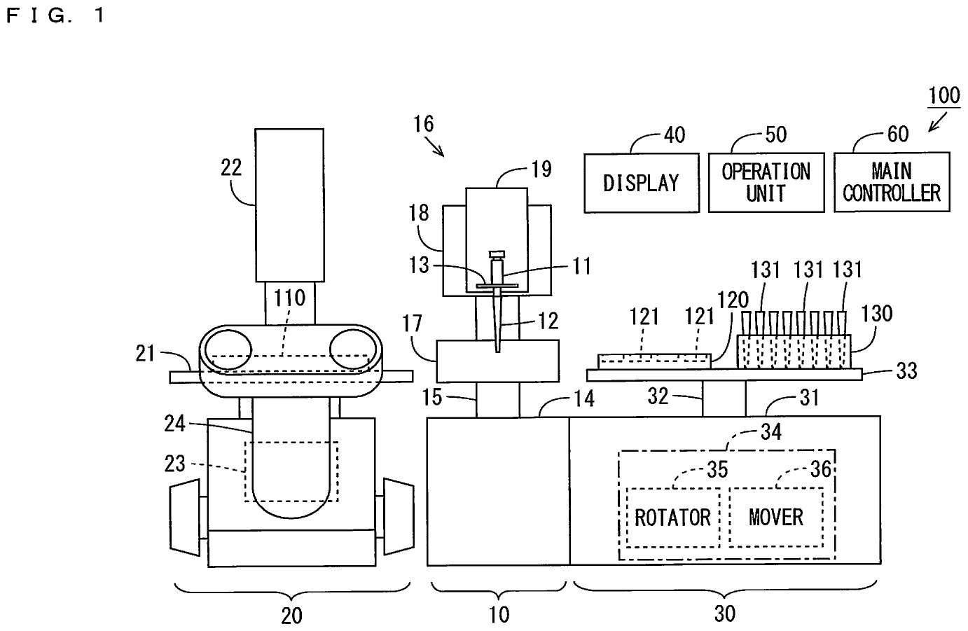

FIG. 1 is a diagram showing the configuration of the cell picking device according to one embodiment of the present invention. As shown in FIG. 1, the cell picking device 100 includes a suction device 10, an observation device 20, a plate changer 30, a display unit 40, an operation unit 50 and a main controller 60. Further, the cell picking device 100 is provided with a sample container 110, an accommodating plate 120 and a pipette tip rack 130 (hereinafter simply referred to as a rack 130.)

The sample container 110 is a petri plate, for example, and accommodates a sample including cells. The accommodating plate 120 is a multi-well plate in which a plurality of wells are arranged and used for culturing of cells. The rack 130 holds a plurality of replacement pipette tips 131 (hereinafter simply referred to as replacement tips 131.) In the present example, 96 wells 121 are arranged in 8 rows by 12 columns in the accommodating plate 120. Further, 96 replacement tips 131 are held while being arranged in 8 rows by 12 columns in the rack 130.

The suction device 10 includes a suction discharger 11, a pipette tip 12 (hereinafter simply referred to as a tip 12,) a holder 13, a base 14, a vertical shaft 15 and a driver 16. The driver 16 includes rotators 17, 18 and a suction driver 19. The suction discharger 11 is a pipette-shape arm. The tip 12 is attached to the end of the suction discharger 11. Any replacement tip 131 held in the rack 130 can be attached to the end of the suction discharger 11 as the tip 12.

The holder 13 holds the suction discharger 11 at the suction driver 19. The vertical shaft 15 is provided on the upper surface of the base 14 to extend in an up-and-down direction. The rotator 17 includes an electric motor, for example, and is attached to the upper end of the vertical shaft 15 to be rotatable in a horizontal plane. The rotator 18 includes an electric motor, for example, and is attached to the rotator 17 to be rotatable in a vertical plane. The rotator 17 and the rotator 18 may be constituted by a single electric motor, etc. which is rotatable in the horizontal plane and the vertical plane.

The suction driver 19 includes a stepping motor, for example, and is attached to the rotator 18 to be advanceable and retreatable in a predetermined direction (an up-and-down direction in a case where the rotator 18 is not rotating in the vertical plane). Further, the suction driver 19 includes a suction mechanism and a discharge mechanism, and is configured to be capable of sucking and discharging cells from the suction discharger 11. Further, the suction driver 19 includes a tip removal mechanism and is configured to be capable of removing the tip 12 from the end of the suction discharger 11.

The driver 16 can scrape off cells in the sample container 110 using the end of the tip 12 and can suck the scraped cells into the tip 12 through the suction discharger 11. Hereinafter, suction of cells in the sample container 110 into the tip 12 is referred to as cell-sucking work. Further, the driver 16 can discharge cells that have been sucked into the tip 12 through the suction discharger 11 into any well 121 in the accommodating plate 120. Hereinafter, discharging of cells that have been sucked into the tip 12 into any well 121 in the accommodating plate 120 is referred to as cell-discharging work. Thus, cells are seeded in any well 121. Cell picking work includes cell-sucking work and cell-discharging work.

With these configurations, in a case where the suction device 10 performs the cell-sucking work, the rotator 17 is rotated in a horizontal plane such that the suction discharger 11 and the tip 12 are directed toward the observation device 20. Further, in a case where the suction device 10 performs the cell-discharging work, the rotator 17 rotates in a horizontal plane such that the suction discharger 11 and the tip 12 are directed toward the plate changer 30.

The observation device 20 includes a stage 21, an illuminator 22, an imager 23 and a microscope 24 and is arranged to be adjacent to the suction device 10. The sample container 110 is placed on the stage 21. The illuminator 22 is arranged above the stage 21. The illuminator 22 includes a light source such as a light emitting diode and illuminates the sample container 110 placed on the stage 21. The stage 21 is translucent. Alternatively, an opening through which light from the illuminator 22 passes downwardly may be formed in the stage 21.

The imager 23 is arranged below the stage 21. The imager 23 includes a plurality of lenses, a camera and so on, and picks up an image while magnifying a sample in the sample container 110 illuminated by the illuminator 22. The microscope 24 includes an eyepiece, a lens-barrel, an objective lens, etc., and is used by a user when a sample in the sample container 110 placed on the stage 21 is magnified for observation.

The plate changer 30 is arranged to be opposite to the observation device 20 with the suction device 10 provided therebetween and is configured to be attachable to and detachable from the suction device 10. The plate changer 30 includes a base 31, a vertical shaft 32, a platform 33 and a driver 34. The vertical shaft 32 is provided to extend in the up-and-down direction in the base 31. An upper portion of the vertical shaft 32 projects from the base 31. The platform 33 is attached to the upper end of the vertical shaft 32 in a horizontal attitude. The accommodating plate 120 and the rack 130 are placed on the platform 33.

The driver 34 includes a rotator 35 and a mover 36, and is connected to the platform 33 through the vertical shaft 32 in the base 31. The rotator 35 includes an electric motor, for example, and rotates the platform 33 in a horizontal plane. Thus, the accommodating plate 120 and the rack 130 placed on the platform 33 are selectively moved to the vicinity of the suction device 10.

Specifically, when the suction device 10 discharges cells into a well 121, the accommodating plate 120 is moved to the vicinity of the suction device 10. When any replacement tip 131 is attached to the suction discharger 11, the rack 130 is moved to the vicinity of the suction device 10. With this configuration, a movement range of the platform 33 is prevented from being increased.

The mover 36 includes a stepping motor, for example, and moves the platform 33 in parallel in a horizontal plane. Specifically, the mover 36 moves a well 121 selected by the user in the accommodating plate 120 to a position accessible by the suction discharger 11 (below the suction discharger 11, for example). Thus, the cell-discharging work can be performed. Hereinafter, a well 121 that has been elected by the user is referred to as a selection well. Cells are accommodated in a selection cell by cell-discharging work. Details of a selection well will be described below.

Further, the mover 36 moves any replacement tip 131 in the rack 130. Thus, a replacement tip 131 can be attached to the suction discharger 11.

The display unit 40 includes an LCD (Liquid Crystal Display) panel or an organic EL (Electroluminescence) panel, for example. The display unit 40 sequentially displays a plurality of GUIs (Graphical User Interfaces) for receiving selection of work contents of the suction device 10, the observation device 20 and the plate changer 30 as selection screens. Further, the display unit 40 displays a work screen for receiving an instruction during work of the cell picking device 100.

The operation unit 50 includes a keyboard, a pointing device, etc. As a pointing device, a mouse, a joy stick or the like is used. The display unit 40 and the operation unit 50 may be integrally constituted by a touch panel display. The user can operate a button, a pull-down menu or the like in a selection screen or a work screen displayed in the display unit 40 using the operation unit 50.

The main controller 60 includes a personal computer, for example, and includes a CPU (Central Processing Unit), a memory and so on. The main controller 60 registers a work procedure (hereinafter referred to as a method) including work contents received by the selection screen. Further, the main controller 60 controls the work of the suction device 10 and the plate changer 30 in accordance with a registered method. The main controller 60 may control the observation device 20 in accordance with a method.

### (2) Creation of Method

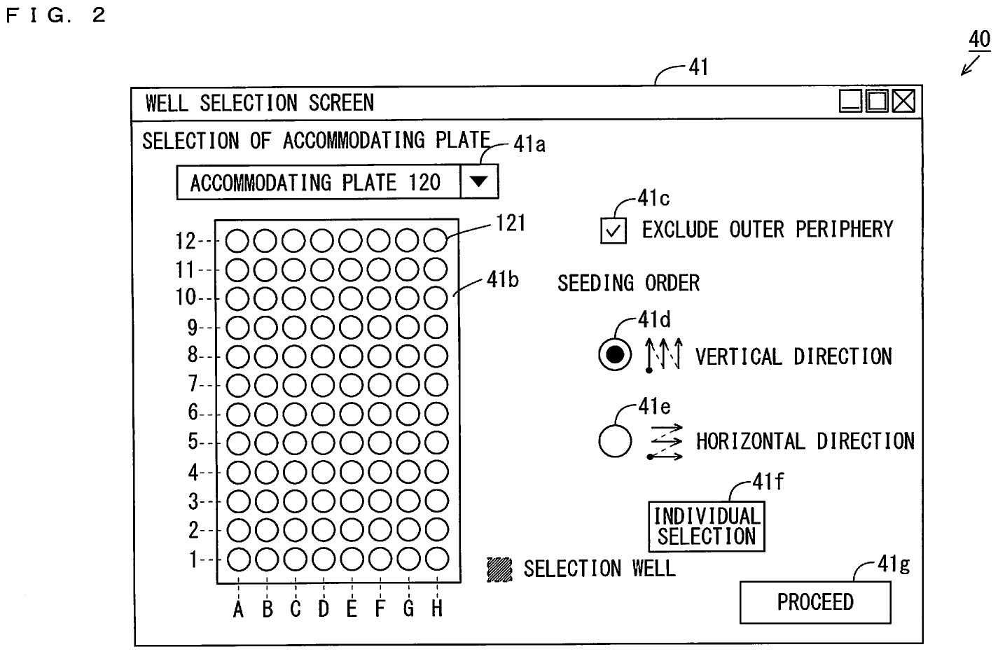

FIGS. 2 to 4 are schematic diagrams showing examples of a selection screen to be displayed in the display unit 40 of FIG. 1 during creation of method. In FIG. 2, a well selection screen 41 is shown. The well selection screen 41 is used for selection of a well in which cells are to be accommodated by cell-discharging work.

As shown in FIG. 2, in the well selection screen 41, a pull-down menu 41a, an accommodating plate display region 41b, a check field 41c, selection fields 41d, 41e, an individual selection button 41f and a proceed button 41g are displayed.

The user selects the type of the accommodating plate of FIG. 1 from the types of accommodating plates displayed in the pull-down menu 41a. In the present example, as shown in FIG. 2, the accommodating plate 120 is selected. Arrangement of wells 121 in each accommodating plate displayed in the pull-down menu 41a is stored.

In the accommodating plate display region 41b, arrangement of the wells 121 in the accommodating plate 120 selected in the pull-down menu 41a is displayed. As displayed in the accommodating plate display region 41b, the plurality of wells 121 in the accommodating plate 120 are arranged in 8 columns in parallel with a first direction (a vertical direction in the present example) by 12 rows in parallel with a second direction (a horizontal direction in the present example) orthogonal to the first direction. Hereinafter, a plurality of rows of the wells 121 are referred to as rows 1 to 12 from the bottom to the top, and a plurality of columns of the wells 121 are referred to as columns A to H from the left to the right.

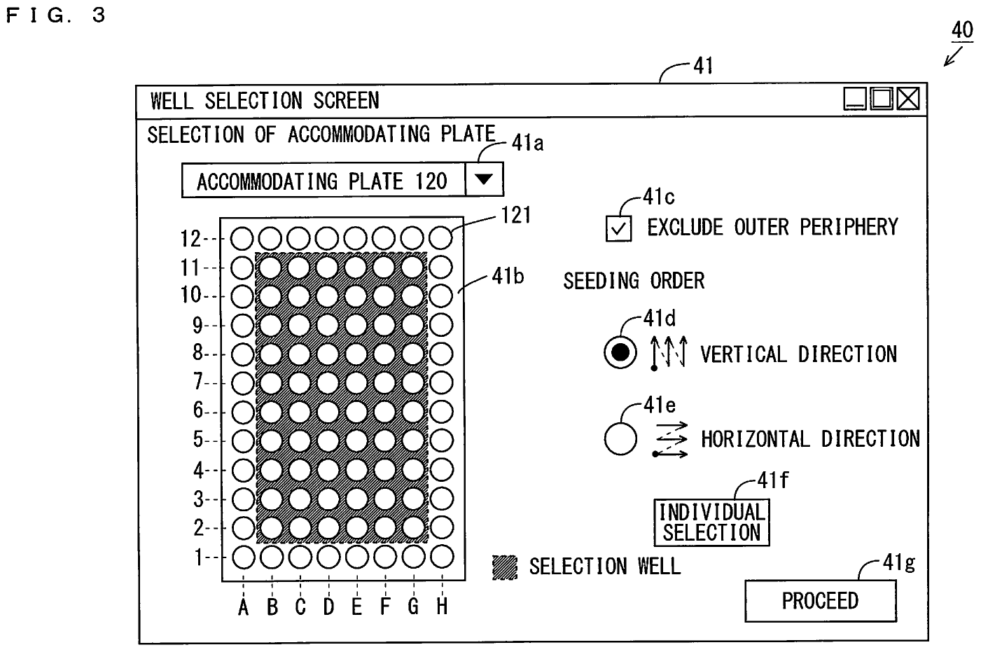

The user checks the check field 41c in a case where wishing to exclude the wells on the outermost periphery from wells subject to cell-discharging work out of the plurality of wells 121 displayed in the accommodating plate display region 41b. When the check field 41c is checked, the display of the accommodating plate display region 41b is switched to the display of the accommodating plate display region 41b shown in FIG. 3. In this case, the plurality of wells 121 positioned on the outermost periphery out of the plurality of wells 121 in the accommodating plate 120 are excluded from selection wells. Further, a plurality of wells 121 other than the plurality of wells 121 positioned on the outermost periphery are selected as selection wells. In the example of FIG. 3, the selection wells are surrounded by a rectangular with hatching. In the actual well selection screen 41, the selection wells are surrounded by a rectangular with a specific color. Thus, the user can distinguish the selection wells from wells not to be selected (hereinafter referred to as non-selection wells.) Selection wells may be displayed under a different luminance from that of non-selection wells, and selection wells may blink. Selections wells may be displayed in another manner to be distinguishable from non-selection wells.

Hereinafter, the order of discharging (seeding) cells into wells 121 by cell-discharging work is referred to as seeding order. The user selects the selection field 41d or 41e in order to determine the seeding order. The seeding order determined by selection of the selection field 41d is referred to as first order, and the seeding order determined by selection of the selection field 41e is referred to as second order.

In the first order, cells are sequentially discharged into the wells 121 in one column A in the rows 1 to 12, then cells are sequentially discharged into the wells 121 in one column B on the right in the rows 1 to 12, and then cells are sequentially discharged into the wells 121 in respective columns (columns C to H) further on the right in the rows 1 to 12.

In the second order, cells are sequentially discharged into the wells 121 in one row 1 in the columns A to H, then cells are sequentially discharged into the wells 121 in one row 2 above in the columns A to H, and then cells are sequentially discharged into the wells 121 in respective rows (rows 3 to 12) further above in the columns A to H.

In the example of FIG. 2, the selection field 41d (the first order) out of the selection fields 41d and 41e is selected. With the above-mentioned setting, the selection wells and the seeding order with respect to the selection wells are determined. In this state, when the user operates the proceed button 41g, the selection wells and the seeding order in the well selection screen 41 are confirmed. Other work procedures are sequentially set, so that a method representing the work procedure of cell-sucking work and cell-discharging work is created. A created method is stored in a storage medium such as a memory.

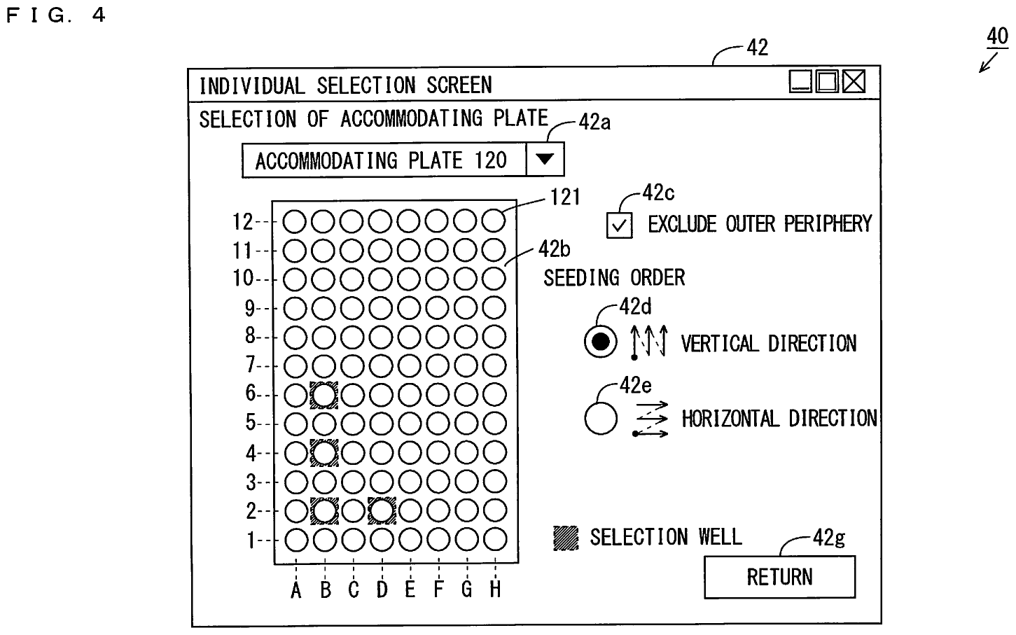

Here, in the well selection screen 41 of FIG. 3, the individual selection button 41f may be operated for individual selection of a selection well out of the plurality of wells in the accommodating plate 120. When the individual selection button 41f is operated, the display in the display unit 40 is switched to an individual selection screen 42 shown in FIG. 4. The individual selection screen 42 is used for individual selection of a well 121 into which cells are to be discharged by cell-discharging work as a selection well.

In an accommodating plate display region 42b in the individual selection screen 42, any one well 121 or a plurality of any wells 121 can be selected out of a plurality of wells 121. In the present example, because the check field 42c is checked, any one well 121 or a plurality of any wells 121 except for the wells positioned at the outermost periphery out of the plurality of wells 121 can be selected as selection wells. In the example of FIG. 4, the well 121 at the intersection of the row 2 and the column B, the well 121 at the intersection of the row 4 and the column B, the well 121 at the intersection of the row 6 and the column B and the well 121 at the intersection of the row 2 and the column D are selected as the selection wells. Also in this case, similarly to the well selection screen 41 of FIG. 3, the selection wells are displayed to be distinguishable from the non-selection wells. For example, the user can select any one well 121 or a plurality of any wells 121 in the accommodating plate display region 42b by clicking a mouse button.

Further, in the example of FIG. 4, because the selection field 42d is selected, cells are to be seeded in the selection well at the intersection of the row 2 and the column B, the selection well at the intersection of the row 4 and the column B, the selection well at the intersection of the row 6 and the column B and the selection well at the intersection of the row 2 and the column D in this order. In the individual selection screen 42 of FIG. 4, in a case where the selection field 42e is selected, cells are to be seeded in the selection well at the intersection of the row 2 and the column B, the selection well at the intersection of the row 2 and the column D, the selection well at the intersection of the row 4 and the column B and the selection well at the intersection of the row 6 and the column B in this order.

When the user operates the proceed button 42g, the selection wells and the seeding order in the individual selection screen 42 are confirmed. Other work procedures are sequentially set, so that a method representing the work procedure of cell-sucking work and cell-discharging work is created. A created method is stored in a storage medium such as a memory. When the user operates a return button (not shown), the display in the display unit 40 switches to the well selection screen 41 from the individual selection screen 42.

The same well 121 can be selected multiple times in the well selection screen 41 of FIG. 3 and the individual selection screen 42 of FIG. 4. In this case, cell-sucking work and cell-discharging work are repeated multiple times with respect to a well that has been selected multiple times. For example, the user can select a well multiple times by clicking on the desired well 121 multiple times in the well selection screen 41 of FIG. 3 or the individual selection screen 42 of FIG. 4.

### (3) Functional Configuration of Main Controller 60

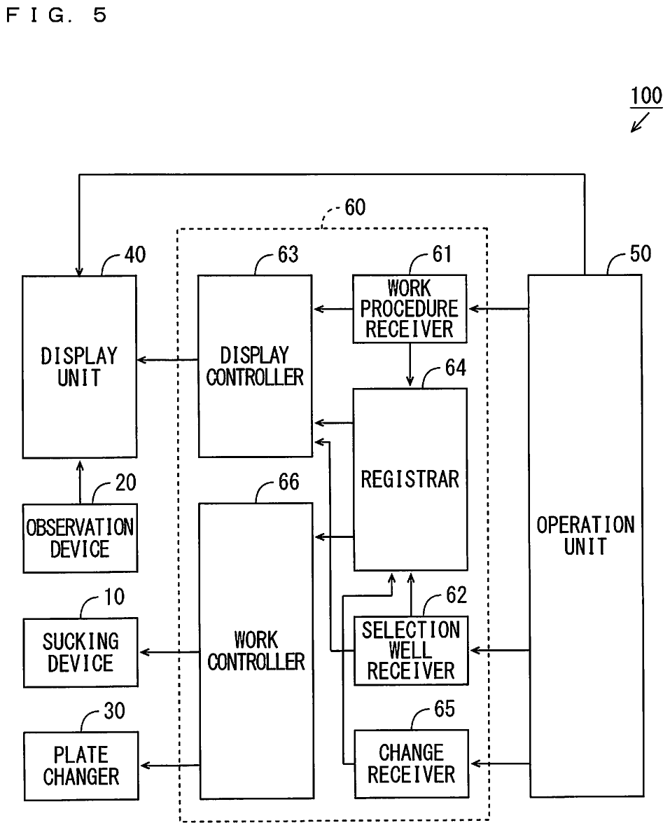

FIG. 5 is a block diagram showing the functional configuration of the main controller 60. As shown in FIG. 5, the main controller 60 includes a work procedure receiver 61, a selection well receiver 62, a display controller 63, a registrar 64, a change receiver 65 and a work controller 66 as functions. The functions of the main controller 60 are implemented by execution of a cell accommodating program stored in a memory by a CPU of the main controller 60. Part or all of the functions of the main controller 60 may be realized by hardware such as an electronic circuit.

The work procedure receiver 61 receives a method that has been created by the user through the display unit 40 and the operation unit 50. The selection well receiver 62 receives one or a plurality of selection wells and seeding order selected by the user in the well selection screen 41 of FIG. 3 or the individual selection screen 42 of FIG. 4.

The display controller 63 displays a plurality of GUIs (Graphical User Interfaces) for receiving selection of the work contents of the suction device 10, the observation device 20 and the plate changer 30 in the display unit 40 as selection screens. Further, the display controller 63 displays a work screen for receiving an instruction during work of the cell picking device 100 in the display unit 40.

The registrar 64 registers a method received by the work procedure receiver 61, and a selection well and seeding order received by the selection well receiver 62. In the present embodiment, a selection well and seeding order are included in a method. In the present example, a method is registered in the memory of the main controller 60.

The change receiver 65 receives a change of a selection well that has been registered in the registrar 64 during work of the cell picking device 100 through the display unit 40 and the operation unit 50 from the user. Further, the registrar 64 registers the change of a selection well received by the change receiver 65.

The work controller 66 controls the work of the suction device 10 and the plate changer 30 in accordance with a method registered in the registrar 64. The work controller 66 may control the observation device 20 in accordance with a method registered in the registrar 64. In particular, the work controller 66 controls the driver 34 of FIG. 1 in the plate changer 30 during cell-discharging work. The work controller 66 may control the work of the suction device 10, the observation device 20 and the plate changer 30 in accordance with a user's operation using the display unit 40 and the operation unit 50.

### (4) Work of Cell Picking Device

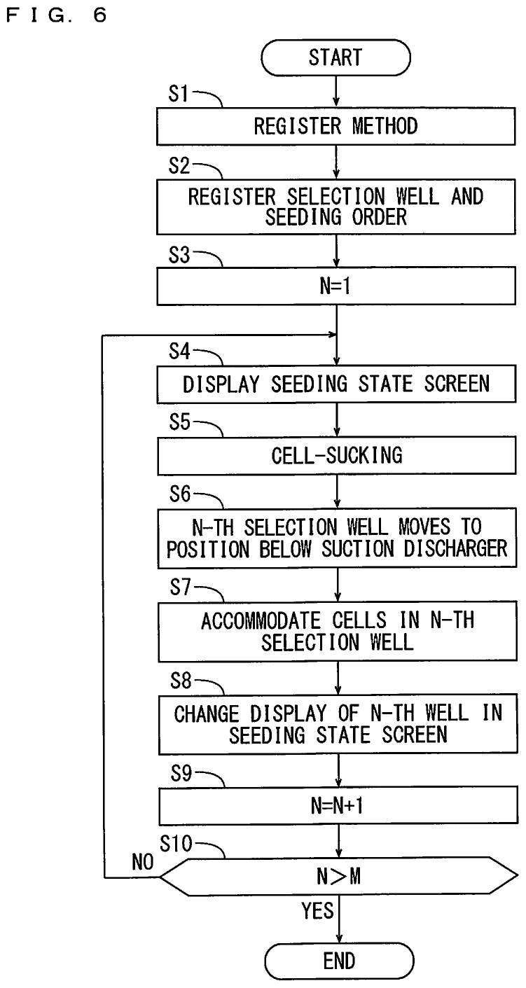

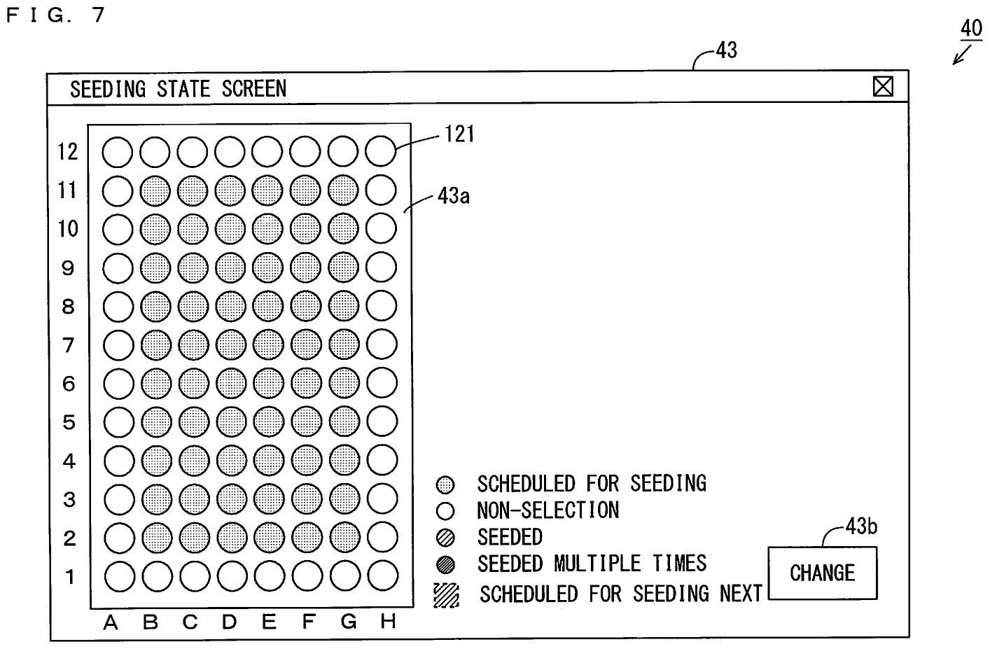

FIG. 6 is a flowchart showing the work of the cell picking device 100 of FIG. 3. FIGS. 7 to 9 are schematic diagrams showing one example of a seeding state screen during picking work.

First, the work procedure receiver 61 receives a method that has been created through the display unit 40 and the operation unit 50 and registers the received method in the registrar 64 (step S1). The selection well receiver 62 receives a selection well and seeding order through the display unit 40 and the operation unit 50 and registers the received selection well and the received seeding order in the registrar 64 (step S2). In the present embodiment, the selection well and the seeding order are included in a method as part of the method.

One or a plurality of selection wells are provided with numbers N (N is an integer) in accordance with the seeding order. Here, the total number of one or a plurality of selection wells is M. The work controller 66 initially sets the number N of a selection well to 1 at the start of picking work (step S3). When picking work is started, the display controller 63 displays the seeding state screen 43 shown in FIG. 7 in the display unit 40 (step S4).

As shown in FIG. 7, an accommodating plate display region 43a and a change button 43b are displayed in the seeding state screen 43. In the accommodating plate display region 43a, the arrangement of the wells 121 in the accommodating plate 120 is displayed, and the seeding state with respect to selection cells by cell-discharging work is displayed in real time. Specifically, a well that has not been selected as a selection well out of the wells 121 in the accommodating plate 120 is displayed without color (in white) as a non-selection well. Hereinafter, a selection well that is scheduled to accommodate cells is referred to as a selection well scheduled for seeding.

For example, a selection well scheduled for seeding is displayed in a specific light color. In FIG. 7, a dotted pattern is applied to a selection well scheduled for seeding.

Subsequently, the work controller 66 controls the suction device 10 such that the cell-sucking work is performed in accordance with a method registered in the registrar 64 (step S5). Thus, cells are sucked into the tip 12.

Thereafter, the work controller 66 controls the suction device 10 and the plate changer 30 such that the cell-discharging work is performed in accordance with the method registered in the registrar 64. Specifically, the work controller 66 controls the driver 34 of the plate changer 30 such that an N-th selection well out of selection wells included in the method registered in the registrar 64 is moved to a position below the suction discharger 11.

In this state, the work controller 66 controls the suction device 10 such that cells that have been sucked into the tip 12 are discharged. Thus, cells are accommodated in the N-th selection well (step S7).

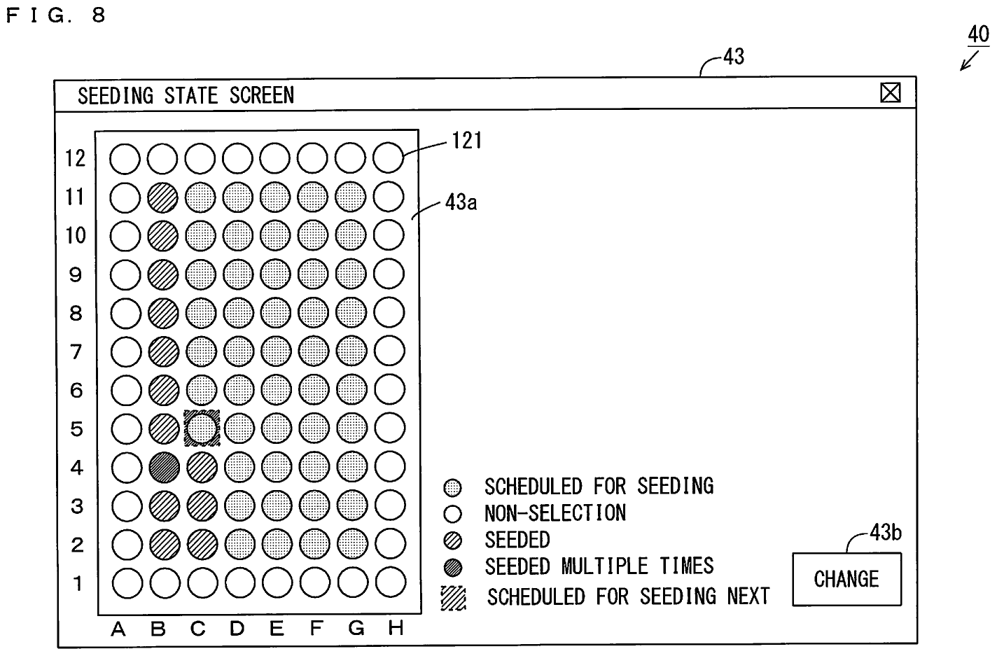

Next, the display controller 63 changes the display of the N-th well 121 in the accommodating plate display region 43a of the seeding state screen 43 of FIG. 7 (step S8). A selection well in which cells have been accommodated by cell-discharging work out of the selection wells is displayed as a selection well in which cells have been seeded in a color different from the color of a selection well scheduled for seeding. In FIG. 8, a hatching pattern is applied to selection wells in which cells have been seeded.

A selection well in which cells have been accommodated by multiple cell-discharging work out of the selection wells in which cells have been seeded is displayed as a selection well in which cells have been seeded multiple times in a color darker than the color of the selection well in which cells have been seeded once. In FIG. 8, a hatching pattern different from the hatching pattern applied to the selection wells in which cells have been seeded once is applied to the selection well in which cells have been seeded multiple times. A selection well in which cells are to be accommodated next by cell-discharging work is surrounded by a rectangular in a specific color as a selection well scheduled for seeding next.

The selection wells in which cells have been seeded may be displayed under different luminance from luminance under which selection wells scheduled for seeding are displayed, and the selection wells in which cells have been seeded may blink. A selection well in which cells have been seeded may be displayed in another manner to be distinguishable from a selection well scheduled for seeding. Further, a selection well in which cells have been seeded multiple times may be displayed in another manner to be distinguishable from a selection well in which cells have been seeded once. Further, the selection well scheduled for seeding next may be displayed in another manner to be distinguishable from the selection wells in which cells have been seeded and the selection well in which cells have been seeded multiple times. In FIG. 7, the seeding state after cells are accommodated in the 13th (N=13) selection well is shown.

Next, the work controller 66 adds 1 to the number N of the selection well (step S9). Thereafter, the work controller 66 determines whether the number N of the selection well is larger than the total number M (step S10). In a case where the number N of the selection well is larger than the total number M, the work controller 66 ends cell-sucking work and cell-discharging work.

In a case where the number N of the selection well is equal to or smaller than the total number M, the work controller 66 returns to the step S4. Thus, the process from the step S4 to the step S8 is repeated until the number N of the selection well exceeds the total number M. Thus, the cell-sucking work and the cell-discharging work are performed with respect to the first to the M-th selection wells.

### (5) Change of Well for Seeding

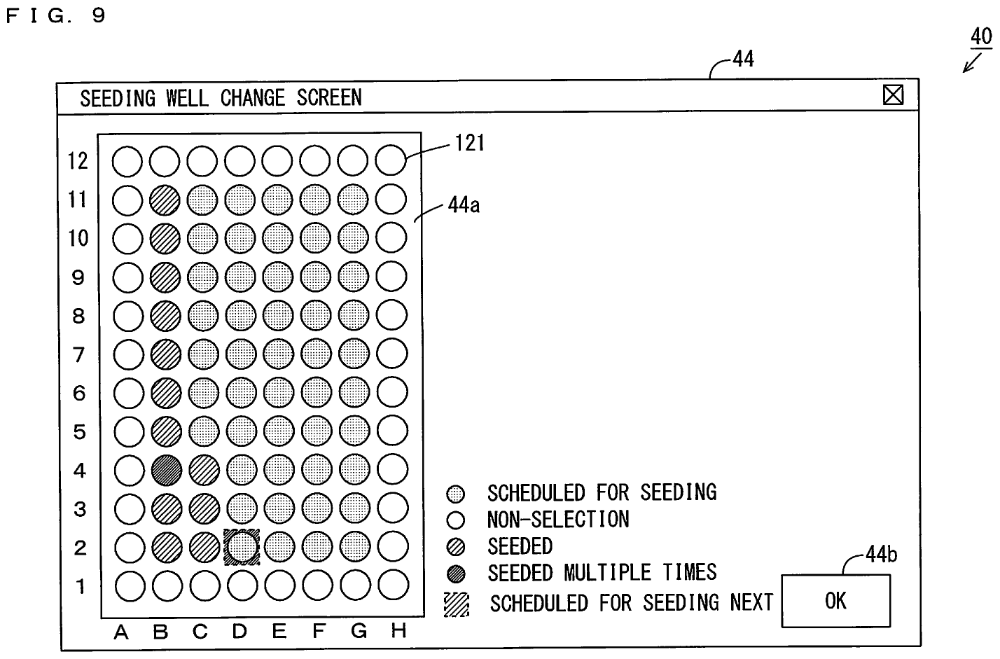

In the above-mentioned example, the user can operate the change button 43b in the seeding state screen 43 of FIG. 8 during cell-picking work. When the change button 43b is operated, the display in the display unit 40 is switched from the seeding state screen 43 to a seeding well change screen 44 of FIG. 9.

The seeding well change screen 44 includes an accommodating plate display region 44a and an OK button 44b. The accommodating plate display region 44a has the display contents similar to that of the accommodating plate display region 43a. Further, the accommodating plate display region 44a is configured such that any selection well scheduled for seeding next is selectable. Here, the user can change a selection well scheduled for seeding next by operating the operation unit 50.

For example, in the seeding state screen 43 of FIG. 8, a selection well scheduled for seeding next is the selection well at the intersection of in the row 5 and the column C. In the seeding well change screen 44 of FIG. 9, the user can change the selection well scheduled for seeding next to the selection well at the intersection of the row 2 and the column D. In this state, when the user operates the OK button 44b, the well scheduled for seeding next is changed to the selection well at the intersection of the row 2 and the column D.

In the present example, the check field 42c and the selection field 42d are selected in the well selection screen 41 of FIG. 3 or the individual selection screen 42 of FIG. 4. Thus, after cells are accommodated in the selection well at the intersection of the row 4 and the column C, the selection wells at the intersection of the row 5 and the column C to the intersection of the row 11 and the column C are skipped, and cells are accommodated in the selection well at the intersection of the row 2 and the column D.

### (6) Effects of Embodiments

With the cell picking device 100 of the present embodiment, a well 121 in which cells are to be accommodated out of the plurality of wells 121 in the accommodating plate 120 is selected. A well 121 that has been selected is received as a selection well. The relative positional relationship between the suction discharger 11 and the plurality of wells 121 is adjusted by the driver 34 such that cells are discharged into the received selection well. Thus, cells can be accommodated automatically in the selection well that has been selected in advance by the user. This alleviates a work load on the user when cells are accommodated in a desired well 121 in the accommodating plate 120.

Further, cells can be automatically accommodated in pre-designated order in selection wells that have been selected in advance by the user. This further alleviates a work load on the user when cells are accommodated in a plurality of desired wells 121 in the accommodating plate 120.

Further, when the user selects one of the predetermined first order and second order by selecting the selection fields 41d, 42d, 41e, 42e in the well selection screen 41 and the individual selection screen 42, cells can be automatically accommodated in selection wells in the selected first order or second order. This facilitates designation of order in regard to a plurality of wells by the user.

Further, in a case where cells are not to be accommodated in a plurality of wells positioned at the outermost periphery, the user can easily avoid accommodation of cells in the plurality of wells positioned at the outermost periphery by checking the check fields 41c, 42d in the well selection screen 41 and the individual selection screen 42.

Further, cells are automatically discharged into selection wells in accordance with the seeding order included in a method registered in the registrar 64. Further, cells can be repeatedly accommodated in selection wells in accordance with a registered method. This further alleviates a work load on the user.

Further, the user can individually designate a selection well in the accommodating plate display region 42b of the individual selection screen 42. Therefore, the user can select a well 121 in which cells are to be accommodated with a simple operation.

Further, a selection well in which cells have been seeded is displayed to be distinguishable in the accommodating plate display region 43a of the seeding state screen 43. Thus, the user can visually identify the accommodating state of cells in one or a plurality of wells 121 of the accommodating plate 120.

Further, the user can designate or change a well 121 into which cells are to be discharged next in the accommodating plate display region 44a of the seeding well change screen 44 during cell-discharging work. This enhances flexibility of automatic cell-discharging work.

Further, the user can visually identify a well 121 into which cells have been discharged multiple times in the accommodating plate display region 43a of the seeding state screen 43 and the accommodating plate display region 44a of the seeding well change screen 44. Thus, the user can easily recognize a well having a high concentration of cells.

### (7) Other Embodiments

While the seeding order in regard to the wells 121 is selectable with use of the selection fields 41d, 41e in the well selection screen 41 and the individual selection screen 42 in the above-mentioned embodiment, the present invention is not limited to this. For example, any seeding order may be settable in the well selection screen 41 and the individual selection screen 42.

While the first order or the second order is selected as the seeding order by selection of one of the selection fields 41d, 41e in the well selection screen 41 and the individual selection screen 42 in the above-mentioned embodiment, three or more seeding order may be selectable in the well selection screen 41 and the individual selection screen 42.

While the check field 41c for excluding a plurality of wells 121 positioned at the outermost periphery of the accommodating plate 120 from selection wells is provided in the well selection screen 41 and the individual selection screen 42, the present invention is not limited to this. For example, a check field for excluding one or a plurality of wells in another region from selection wells may be provided.

### (8) Correspondences Between Constituent Elements in Claims and Parts in Preferred Embodiments

In the following paragraphs, non-limiting examples of correspondences between various elements recited in the claims below and those described above with respect to various preferred embodiments of the present disclosure are explained.

In the above-mentioned embodiment, the selection well receiver 62 is an example of a receiver, the accommodating plate display region 42b is an example of a first well image, and the accommodating plate display region 43a and the accommodating plate display region 44a are examples of a second well image.

### (9) Aspects

It is understood by those skilled in the art that the plurality of above-mentioned illustrative embodiments are specific examples of the below-mentioned aspects.

(Item 1) A cell picking device according to one aspect sucks cells from a liquid sample in a sample container and accommodates the sucked cells in any of a plurality of wells of an accommodating plate, may include a suction discharger that sucks cells in the sample container and discharges the sucked cells into any of the plurality of wells, the receiver that receives selection of a well in which cells are to be accommodated out of the plurality of wells of the accommodating plate as a selection well, a driver that adjusts a relative positional relationship between the suction discharger and the plurality of wells of the accommodating plate, and a work controller that controls the driver such that the suction discharger discharges cells into the received selection well out of the plurality of wells of the accommodating plate.

With the cell picking device according to one aspect, selection of a well in which cells are to be accommodated out of a plurality of wells is received as a selection well. The relative positional relationship between the suction discharger and the plurality of wells are adjusted such that cells are to be discharged into the received selection well. Thus, cells can be automatically accommodated in the well that has been selected in advance by the user. This alleviates a work load on the user when cells are accommodated in a desired well of the accommodating plate.

(Item 2) The cell picking device according to item 1, wherein the receiver may be configured to be capable of receiving selection of a plurality of wells in which cells are to be accommodated as a plurality of the selection wells and receiving discharging order of cells into the plurality of the received selection wells, and the work controller may control the driver such that the suction discharger discharges cells into the plurality of the selection wells in the discharging order.

With the cell picking device according to the item 2, cells can be automatically accommodated in a plurality of wells that have been selected in advance by the user in the pre-designated order. This further alleviates a work load on the user when cells are accommodated in the plurality of desired wells of the accommodating plate.

(Item 3) The cell picking device according to item 2, wherein the receiver may be configured to be capable of selecting one of first order and second order that are different from each other as the discharging order.

With the cell picking device according to the item 3, when the user selects one of the predetermined first order and second order, cells can be automatically accommodated in a plurality of wells in the selected order. This facilitates designation of order in regard to a plurality of wells by the user.

(Item 4) The cell picking device according to item 3, wherein the plurality of wells of the accommodating plate may be arranged in a plurality of columns in parallel with a first direction and arranged in a plurality of rows in parallel with a second direction intersecting with the first direction, the first order may include order in which cells are sequentially discharged into a plurality of wells arranged in one column, and then cells are sequentially discharged into a plurality of wells arranged in another adjacent column, and the second order may include order in which cells are sequentially discharged into a plurality of wells arranged in one row and then cells are sequentially discharged into a plurality of wells arranged in another adjacent row.

With the cell picking device according to item 4, it is possible to easily and sequentially accommodate cells in a plurality of wells arranged in each row and sequentially accommodate cells in a plurality of wells arranged in each column by selection easily made by the user.

(Item 5) The cell picking device according to any one of items 1 to 4, wherein the receiver may be configured to be capable of performing a setting such that one or a plurality of wells out of the plurality of wells of the accommodating plate are not to be selected as the selection wells.

With the cell picking device according to item 5, selection of one or a plurality of wells in which cells are not to be accommodated as selection wells can be prevented. In this case, accommodation of cells in one or a plurality of wells in which cells are not to be accommodated can be easily avoided.

(Item 6) The cell picking device according to any one of items 1 to 5, wherein the receiver may be configured to be capable of performing a setting such that a plurality of wells positioned at an outermost periphery out of the plurality of wells of the accommodating plate are not to be selected as the selection wells.

With the cell picking device according to item 6, in a case where a plurality of wells positioned at the outermost periphery are not to accommodate cells, selection of the plurality of wells positioned at the outermost periphery as selection wells can be prevented. In this case, accommodation of cells in the plurality of wells positioned at the outermost periphery can be easily avoided.

(Item 7) The cell picking device according to any one of items 1 to 6, may further include a registrar that registers a work procedure of cell-discharging work of the suction discharger and a selection well that has been received by the receiver, wherein the work controller may control the driver such that the suction discharger discharges cells into the registered selection well in accordance with the work procedure registered in the registrar.

With the cell picking device according to item 7, cells are automatically discharged into selection wells that have been registered in accordance with the pre-registered work procedure. Further, cells can be accommodated repeatedly in the selection wells in accordance with the registered work procedure. This further alleviates a work load on the user.

(Item 8) The cell picking device according to any one of items 1 to 6, may further include a display controller that displays an image showing the plurality of wells of the accommodating plate as a first well image, wherein the receiver may be configured to be capable of selecting the selection well on the first well image.

With the cell picking device according to item 8, the user can designate a selection well on the first well image. Therefore, the user can select a well in which cells are to be accommodated with a simple operation.

(Item 9) The cell picking device according to item 8, wherein the display controller may display an image showing the plurality of wells of the accommodating plate in the display unit as a second well image and may distinguishably display a well in which cells are accommodated on the second well image based on control of the work controller.

With the cell picking device according to claim 9, a well in which cells are accommodated is distinguishably displayed on the second well image. Thus, the user can visually identify the accommodating state of cells in one or a plurality of wells of the accommodating plate.

(Item 10) The cell picking device according to item 9, wherein the display controller may display the second well image in the display unit such that a well into which cells are to be discharged next is designatable or changeable when the suction discharger are discharging cells into the selection well of the accommodating plate.

With the cell picking device according to claim 10, the user can designate or change a well into which cells are to be discharged next on the second well image during cell-discharging work. This enhances flexibility of automatic cell-discharging work.

(Item 11) The cell picking device according to item 9, wherein the display controller may distinguishably display a well into which cells have been discharged multiple times on the second well image in the display unit in a case where cells have been discharged multiple times into the selection well of the accommodating plate by the suction discharger.

With the cell picking device according to item 11, the user can visually identify a well into which cells have been discharged multiple times. Thus, the user can easily identify that a well has a high concentration of cells.

(Item 12) A cell picking method according to another aspect of sucking cells from a liquid sample in a sample container and accommodating the sucked cells in any of a plurality of wells of an accommodating plate may include receiving selection of a well in which cells are to be accommodated out of the plurality of wells of the accommodating plate as a selection well, sucking cells in the sample container using a suction discharger, adjusting a relative positional relationship between the suction discharger and the plurality of wells of the accommodating plate using a driver such that the cells are dischargeable into the selection well, and discharging cells that have been sucked by the suction discharger into the selection well after adjustment of the relationship by the driver.

With the cell picking method according to another aspect, selection of a well in which cells are to be accommodated out of a plurality of wells is received as a selection well. After the relative positional relationship between the suction discharger and a plurality of wells is adjusted such that cells can be discharged into the accepted selection cell, cells that have been sucked by the suction discharger are discharged into the selection well. Thus, cells can be automatically accommodated in the well that has been selected in advance by the user. This alleviates a work load on the user when cells are accommodated in a desired well of the accommodating plate.

(Item 13) The cell picking method according to item 12, wherein the receiving may include receiving selection of a plurality of wells in which cells are to be accommodated as the plurality of the selection wells and receiving discharging order of cells into the plurality of the received selection cells, and the discharging may include discharging of cells into the plurality of the selection wells in the discharging order by the suction discharger.

With the cell picking method according to item 13, cells can be automatically accommodated in the pre-designated order in a plurality of wells that have been selected in advance by the user. This further alleviates a work load on the user when cells are accommodated in the plurality of desired wells of the accommodating plate.

(Item 14) The cell picking method according to item 13, wherein the receiving may include receiving one of first order and second order that are different from each other as the discharging order.

With the cell picking method according to item 14, when the user selects one of the predetermined first order and second order, cells can be automatically accommodated in a plurality of wells in the selected order. This facilitates designation of order in regard to a plurality of wells by the user.

(Item 15) The cell picking method according to any one of items 12 to 14, wherein the receiving may include receiving a setting in which one or a plurality of wells out of the plurality of wells of the accommodating plate are not selected as the selection wells.

With the cell picking method according to item 15, selection of one or a plurality of wells in which cells are not to be accommodated as selection wells can be prevented. In this case, accommodation of cells in one or a plurality of wells in which cells are not to be accommodated can be easily avoided.

While preferred embodiments of the present disclosure have been described above, it is to be understood that variations and modifications will be apparent to those skilled in the art without departing the scope and spirit of the present disclosure. The scope of the present disclosure, therefore, is to be determined solely by the following claims.

I/We claim:

1. A cell picking device that sucks cells from a liquid sample in a sample container and accommodates the sucked cells in any of a plurality of wells of an accommodating plate, comprising:
a suction discharger that sucks cells in the sample container and discharges the sucked cells into any of the plurality of wells;
the receiver that receives selection of a well in which cells are to be accommodated out of the plurality of wells of the accommodating plate as a selection well;
a driver that adjusts a relative positional relationship between the suction discharger and the plurality of wells of the accommodating plate; and
a work controller that controls the driver such that the suction discharger discharges cells into the received selection well out of the plurality of wells of the accommodating plate.

2. The cell picking device according to claim 1, wherein
the receiver is configured to be capable of receiving selection of a plurality of wells in which cells are to be accommodated as a plurality of the selection wells and receiving discharging order of cells into the plurality of the received selection wells, and
the work controller controls the driver such that the suction discharger discharges cells into the plurality of the selection wells in the discharging order.

3. The cell picking device according to claim 2, wherein
the receiver is configured to be capable of selecting one of first order and second order that are different from each other as the discharging order.

4. The cell picking device according to claim 3, wherein
the plurality of wells of the accommodating plate are arranged in a plurality of columns in parallel with a first direction and arranged in a plurality of rows in parallel with a second direction intersecting with the first direction,
the first order includes order in which cells are sequentially discharged into a plurality of wells arranged in one column, and then cells are sequentially discharged into a plurality of wells arranged in another adjacent column, and
the second order includes order in which cells are sequentially discharged into a plurality of wells arranged in one row and then cells are sequentially discharged into a plurality of wells arranged in another adjacent row.

5. The cell picking device according to claim 1, wherein
the receiver is configured to be capable of performing a setting such that one or a plurality of wells out of the plurality of wells of the accommodating plate are not to be selected as the selection wells.

6. The cell picking device according to claim 1, wherein
the receiver is configured to be capable of performing a setting such that a plurality of wells positioned at an outermost periphery out of the plurality of wells of the accommodating plate are not to be selected as the selection wells.

7. The cell picking device according to claim 1, further comprising a registrar that registers a work procedure of cell-discharging work of the suction discharger and a selection well that has been received by the receiver, wherein
the work controller controls the driver such that the suction discharger discharges cells into the registered selection well in accordance with the work procedure registered in the registrar.

8. The cell picking device according to claim 1, further comprising a display controller that displays an image showing the plurality of wells of the accommodating plate as a first well image, wherein
the receiver is configured to be capable of selecting the selection well on the first well image.

9. The cell picking device according to claim 8, wherein
the display controller displays an image showing the plurality of wells of the accommodating plate in the display unit as a second well image and distinguishably displays a well in which cells are accommodated on the second well image based on control of the work controller.

10. The cell picking device according to claim 9, wherein
the display controller displays the second well image in the display unit such that a well into which cells are to be discharged next is designatable or changeable when the suction discharger are discharging cells into the selection well of the accommodating plate.

11. The cell picking device according to claim 9, wherein
the display controller distinguishably displays a well into which cells have been discharged multiple times on the second well image in the display unit in a case where cells have been discharged multiple times into the selection well of the accommodating plate by the suction discharger.

12. A cell picking method of sucking cells from a liquid sample in a sample container and accommodating the sucked cells in any of a plurality of wells of an accommodating plate; including:
receiving selection of a well in which cells are to be accommodated out of the plurality of wells of the accommodating plate as a selection well;
sucking cells in the sample container using a suction discharger;
adjusting a relative positional relationship between the suction discharger and the plurality of wells of the accommodating plate using a driver such that the cells are dischargeable into the selection well; and
discharging cells that have been sucked by the suction discharger into the selection well after adjustment of the relationship by the driver.

13. The cell picking method according to claim 12, wherein
the receiving includes receiving selection of a plurality of wells in which cells are to be accommodated as the plurality of the selection wells and receiving discharging order of cells into the plurality of the received selection cells, and
the discharging includes discharging of cells into the plurality of the selection wells in the discharging order by the suction discharger.

14. The cell picking method according to claim 13, wherein
the receiving includes receiving one of first order and second order that are different from each other as the discharging order.

15. The cell picking method according to claim 12, wherein
the receiving includes receiving a setting in which one or a plurality of wells out of the plurality of wells of the accommodating plate are not selected as the selection wells.
